(19) United States
(12) Patent Application Publication
(10) Pub. No.: US 2021/0169945 A1
(43) Pub. Date: Jun. 10, 2021

(54) MANUFACTURING OR ONSIDE INSTALLATION OF COMPOSITIONS WITH METHODS, SYSTEMS TO ADDRESS ECOLOGICAL AND ECONOMICAL CONCERNS OF THE AQUACULTURE INDUSTRY

(71) Applicant: Mahalakshmi N.A. SRINIVASAN, Mumbai (IN)

(72) Inventor: Mahalakshmi N.A. SRINIVASAN, Mumbai (IN)

(21) Appl. No.: 17/114,220

(22) Filed: Dec. 7, 2020

Related U.S. Application Data
(60) Provisional application No. 62/945,031, filed on Dec. 6, 2019.

(51) Int. Cl. A61K 35/741; A61K 35/747; A61K 35/744; A61K 35/742; A01K 61/13

(52) U.S. Cl. CPC A61K 35/741; A61K 35/747; A01K 61/13; A61K 35/742; A61K 35/744

(57) ABSTRACT

The present dis sure generally relates to compositions, methods, systems to address stress, starvation, improved health, water quality, productivity and life expectancy of fishes. The composition(s) could comprise of bacteria that could be New Species, Variant of the Known species or Known species. The growth medium is used first to cultivate the bacteria and then added to the incubated bacterial suspension in a dehydrated form to complete the composition. The production unit of the product could be made an integral setup of farms, ponds, companies into transportation of Ornamental fishes, retail outlets. The methodologies involve pretreatment of fishes and introduction of the product into water. The manufactured product could be packaged in containers of different sizes and materials with the shelf life for best to use ranging from one month to six months from the date of manufacturing.

### TECHNICAL FIELD

The present disclosure generally relates to compositions, methods, systems to address stress, starvation, improved health, improved water quality, productivity and life expectancy of fishes. The product addresses the concerns associated with Aquaculture industry, giving better living conditions for the fishes and eliminates the kind of stress, starvations which could be encountered throughout the distribution chain of the Aquaculture industry. The product serves as probiotic, prebiotic, cleanses the water. Also, with this product the water used at aquariums needs to be replaced with lesser frequency and amount of water used for transportation could be lesser than the currently prescribed amounts in the protocols. The compositions, methods, systems enable disease control, improvement of digestion, improvement in immune system, improvement in color, more compatible with other fishes, compositions could serve source of nutrients, growth promotors and promotor of reproduction. These features mentioned are the major characteristics of the composition(s) and each of the composition(s) address the various health parameters associated with the different type of fishes. The composition(s) and the proportions of the components of the composition(s) could be varied to be specific for the fish type. The product has applications throughout the distribution chain of Aquaculture industry, starting from farms or ponds, during transportation, aquariums, wholesalers, retailers, end users. This includes fish farming which could be practiced in Open-net pen and Cage system, Irrigation ditch or pond systems, Inland pond culture, Integrated recycling systems, Flow-through/Raceway or Classic fry farming. The production unit for this product could be made an integral setup of Aquaculture farms, ponds, aquariums. Also, we could supply this product as a manufactured product to farms, ponds, aquariums, wholesalers, companies into transportation of live stock, retail outlets, end users. Based on environment being used, the composition(s), method(s), system(s) could be modulated. The shelf life, packaging process depends upon the components of the composition(s).

### BACKGROUND

Background description includes information that may be useful in understanding the present invention. It is not an admission that any of the information provided herein is prior art to the presently claimed invention, or that any publication specifically or implicitly referenced is prior art.

The invention addresses concerns associated with Aquaculture industry. The product enables close to 0% mortalities; one of the reasons of mortalities being associated with stress and starvation during transportation, at aquariums and farms, the product improves water quality by reducing nitrates, nitrites and ammonia close to 0 ppm and maintaining at that level, water needs to be replaced with lesser frequency and packaging of fishes can happen in transportation bags with lesser amount of water, product serves as a prebiotic and probiotic, composition(s) can be used in different proportions, composition(s) could be modulated in terms of the various components and parameters used in the productions of the composition(s).

The ecologists express concerns about over fishing considering the fact that the actual mortalities associated with the industry aren't revealed. Fishing can lead to affecting the marine ecosystem, reduce density of the fishes in a given ecosystem and alter the balance of the ecosystem which is not due to gradual changes through natural succession.

Fish farming requires that mortalities of fishes is minimized and productivity improved. This invention not only improves productivity but also saves on resources like water and reduces expenses, since not many products are required with this one stop solution being used.

The product gets the mortalities close to 0% and the various parameters of water —nitrates, nitrites and ammonia close to 0 ppm and maintains at that level. Further, this product or technology serves both as a prebiotic and probiotic.

The value of ornamental fish trade has grown significantly over the past decades. Between 2000 and 2011, global exports of ornamental fish increased from US$181 million to US$372 million. Total trade in live marine ornamentals is estimated at around US$44 million annually. Most of the market supplies originate from Asia, with Singapore dominating as the top exporting country in the world. In 2013, Singapore exported around US$56 million worth of ornamental fish to over 80 countries. Ref: GLOBEFISH—Information and Analysis on World Fish Trade. Food and Agriculture Organization of the United Nations.

There are numerous literatures available with regard to the Ornamental fish industry statistics. This Ornamental Fish industry data mentioned here has been provided by ‘Ornamental Fish International (OFI)’. The ornamental fish industry includes more than 120 countries in the collection, breeding, import and export of ornamental fish.

The European Union, collectively, is the largest market for ornamental fish; however, the United States (US) is the single largest importing country for ornamental fish in the world. The prices of fish at the retail level may be several times higher than at the farm gate. This value chain can be extended through additional middlemen who aggregate supplies of fish for export, or who distribute to wholesalers, all of which add to the end cost of fish. The ornamental fish industry is generally segmented based on whether fish are tropical or temperate, freshwater or marine. The three categories are: Tropical Freshwater, Temperate (Coldwater) Freshwater, Marine Ornamentals. The Tropical Freshwater fishes are the most popular, 800 plus species, accounts for more than 50% of trade. (For example: Barbs, Bettas, Mollies, Angelfish). The Temperate (Coldwater) Freshwater accounts for 45% of trade and the majority farmed (For example: Barb, tetra, Danio).

A literature of ‘2019-2024 Global Ornamental Fish Market Trends, Outlook and Consumption Growth Opportunities and Industry, Demand, and Forecast’, 2019, Reuters, states that the worldwide market for Ornamental Fish is expected to grow at a CAGR (Compound Annual Growth Rate) of roughly 6.1% over the next five years, will reach 12000 million US$ in 2024, from 8380 million US$ in 2019, according to a new GIR (Global Info Research) study.

### OBJECTIVES OF THE INVENTION

The objectives of the present disclosure relates to compositions, methods, systems to address stress, starvation, improved health, improved water quality, productivity and life expectancy for fishes. Further, the objectives get into the details of the onsite installation of this technology and manufacturing, usage of the product. The product addresses the concerns associated with Aquaculture industry, giving better living conditions for the fishes and eliminates the kind of stress, starvations they would face throughout the distribution chain of the Aquaculture industry. The product serves as probiotic and prebiotic. Also, with this product the water used at aquariums needs to be replaced with lesser frequency and amount of water used for transportation could be lesser than the currently prescribed amounts in the protocols. There is improvement in color and fishes are more compatible with each other. These features mentioned are some of the major characteristics. Each of the composition(s) could be modulated to be specific for a given fish type. The product has applications throughout the distribution chain, starting from farms or ponds, wholesalers, companies into transportation of live stock, retailers, end users. The production unit for the product could be made an integral setup of Aquaculture farms, ponds or the technology could be availed as a manufactured product with the necessary system(s) provided.

One objective is to develop composition(s) which eliminates stress and starvation associated with fishes, aquatic life forms and provide them as a manufactured product or onsite installation to address ecological and economical concerns of Aquaculture industry,

Another objective is to modulate the composition(s), method(s), system(s) that are environment specific,

Another objective is to develop and use composition(s), method(s), system(s) which are customized based on the environment it is being used,

Another objective is to get mortalities of fishes close to 0% during transportation; Death On Arrival (DOA), Death After Arrival (DAA), Death After 7 days Of Arrival (DA7) to be maintained close to 0%. Also the objective is to maintain mortalities at aquariums, farms and throughout the distribution chain close to 0%,

Another objective is to develop composition(s) which serve as probiotic,

Another objective is to develop composition(s) which serve as prebiotic,

Another objective is to reduce nitrates, nitrites and ammonia build-up and main-taining at low ppm, close to 0 ppm, Another objective is to develop composition(s) with which the fishes could be pretreated,

Another objective is to develop composition(s) by which water needs to be replaced with lesser frequency at farms, aquariums and packaging for transportation can happen with water lesser than the amount prescribed in existent protocols,

Another objective is to develop composition(s) which could be incorporated into Recirculating Aquaculture System (RAS),

Another objective is to develop composition(s) specific for the different types of fishes by fine tuning the proportions of the components of the composition(s),

Another objective is to develop composition(s) which enable fishes to acclimatize in new environments in a very short time,

Another objective is to develop composition(s) such that their production units could be made an integral setup of farms. This requires easy procedures for scaling up in order to cater from the farming stage, through the distribution chain to end user. The technology could also be sold throughout the distribution chain of aquaculture as a manufactured product,

Another objective is modulating the methodology of usage of the composition(s), method(s), system(s) which would be environment specific. This would help define the scaling up procedure depending on the environment it is being used.

Various objects, features, aspects and advantages of the present invention will become more apparent from the detailed description of the invention and accompanying figures.

### SUMMARY

The present disclosure generally relates to compositions, methods, systems to address stress, starvation, improved health, improved water quality, productivity and life expectancy for fishes. The product addresses the concerns associated with Aquaculture industry, giving better living conditions for the fishes and eliminates the kind of stress, starvations they would face throughout the distribution chain of the Aquaculture industry. The product serves as probiotic and prebiotic. Also, with this product the water used at aquariums needs to be replaced with lesser frequency and amount of water used for transportation could be lesser than the currently prescribed amounts in the protocols. There is improvement in color and fishes are more compatible with each other. These features mentioned are the major characteristics of the composition(s) and each of the composition(s) address the various health parameters associated with each type of fishes. The composition(s) could be modulated in terms of the components and their respective proportions, they could be made highly specific for a given fish type. The product has applications throughout the distribution chain, starting from farms or ponds, wholesalers, companies into transportation of live stock, retailers and end users. The production unit for the product could be made an integral setup of Aquaculture farms. An important aspect to be considered is modulating the composition(s), method(s), system(s) which would be environment specific. Here, the environment refers to aquariums, farms, ponds, water bodies, during transportation, at retails, at consumer or end user level; however the environment isn't only restricted to these. These would help define the scaling up procedure depending on the environment it is being used.

There is a wide range of bacteria that could be used in the composition individually or in combination based on the kind of relationship they share (Commensal, Mutualistic, Symbiotic) and their efficacy as a probiotic. These include gram positive bacteria and gram negative bacteria.

The bacteria could be New Species, Variants of known species and the known species.

Some of the species belonging to the following genus of the bacteria could be used in the composition but the scope of the bacteria that could be used isn't restricted to these and these are representative:
Alteromonas, Arthrobacter, Bacillus, Brevibacterium, Hydrogenophaga, Lactobacillus, Leuconostoc, Pediococcus, Phyllobacterium, Pseudoalteromonas, Pseudomonas, Streptomyces, Shewanella.

The species could be as follows but the scope is not restricted to the following and these are representative. The details are as follows:
Alteromonas:
Pseudoalteromonas arabiensis, Pseudoalteromonas luteoviolacea, Pseudoalteromonas rubra, Pseudoalteromonas sp.
Arthrobacter:
Arthrobacter species known to produce therapeutic biomolecules and used based on the fish health condition and fish type.
Bacillus:
Bacillus amyloliquefaciens, Bacillus firmus, Bacillus fiexus, Bacillus lentus, Bacillus pumilus, Bacillus potymyxa, Bacillus megatarium, Bacillus alvei, Bacillus subtilis, Bacillus siamensis, Bacillus sonorensis, Bacillus vallismortis, Bacillus velezensis, Bacillus paralicheniformis, Bacillus vireti
Brevibacterium:
Brevibacterium aureum, Brevibacterium fuscum, Brevibacterium sp.
Hydrogenophaga:
Hydrogenophaga intermedia
Lactobacillus:
Lactobacillus sakei, Lactobacillus rogosae, Lactobacillus plantarum, Lactobacillus pentosus, Lactobacillus paracasei, Lactobacillus panis, Lactobacillus pontis, Lactobacillus reuteri, Lactobacillus fiumenti, Lactobacillus sanfranciscensis, Lactobacillus casei, Lactobacillus coryniformis, Lactobacillus mellis, Lactobacillus brevis, Lactobacillus buchneri, Lactobacillus bulgaricus, Lactobacillus curvatus, Lactobacillus farciminis, Lactobacillus firmentum, Lactobacillus helveticus, Lactobacillis lactis, Lactobacillus reuteri, Lactobacillus johnsonii
Leuconostoc:
Leuconostoc citreum, Leuconostoc pseudomesenteroides, Leuconostoc litchii
Pediococcus:
Pediococcus acidilactici, Pediococcus damnosus, Pediococcus pentosaceus, Pediococcus cerevisiae, Pediococcus argentinicus, Pediococcus cellicola, Pediococcus claussenii, Pediococcus ethanolidurans, Pediococcus inopinatus Pediococcus parvenus, Pediococcus pentosaceus, Pediococcus siamensis, Pediococcus stilesii
Phyllobacterium:
Phyllobacterium brassicacearum, Phyllobacterium qiyense, Phyllobacterium sophorae, Phyllobacterium catacumbae, Phyllobacterium endophyticum, Phyllobacterium qiyense, Phyllobacterium leguminum, Phyllobacterium loti Phyllobacterium myrsinacearum, Phyllobacterium phragmitis, Phyllobacterium rubiacearum, Phyllobacterium salinisoli, Phyllobacterium sophorae, Phyllobacterium trifolii, Phyllobacterium zundukense
Pseudoalteromonas:
Pseudoalteromonas arabiensis, Pseudoalteromonas luteoviolacea, Pseudoalteromonas rubra
Pseudomonas:
Pseudomonas alkylphenolical, Pseudomonas alcaliphila, Pseudomonas bauzanensis, Pseudomonas clemanceaPseudomonas chlororaphis, Pseudomonas carboxydohydrogena, Pseudomonas glycinae, Pseudomonas koreensis Pseudomonas kilonensis, Pseudomonas indica, Pseudomonas lini, Pseudomonas rhizosphaerae, Pseudomonas pachastrellae, Pseudomonas grimontii, Pseudomonas graminis
Shewanella:
Shewanella sediminis
Streptomyces:
Streptomyces violaceoruber, Streptomyces ansochromogenes, Streptomyces aureofaciens, Streptomyces noursei Streptomyces rimosus, Streptomyces sp. S073

One example is the bacterial species identified as New Species belonging to the family Pseudomonadaceae used in the composition(s) individually or as a combination of them in varied proportions. However, the scope of this invention isn't limited to these four New species and other bacteria could be used in the composition(s) which could render these benefits to the Aquaculture industry. Further, as stated earlier, each of the component(s) could be varied in proportion(s) and be specific for a given fish type.

Growth Medium: The growth medium could be Simple, Complex, Selective or Customized medium.

The incubation time for the preparation of the composition depends on the inoculum, preference for the growth medium, growth patterns observed. The parameters for bacterial growth have an important role in the efficacy of the composition(s).

The methodology of preparation basically involves cultivating the bacteria in the growth medium, followed by addition of the dehydrated growth medium to complete the composition preparation. The proportions are dependent on the bacteria and growth medium chosen.

There is a need for Systems to introduce the composition(s) into the biofiltration system of Recirculatory Aquaculture System (RAS), Systems to enable 0 ppm nitrates, nitrites and ammonia at aquariums, Fish Only Tanks and Systems to enable the production of the product to be either packaged and distributed or provided as an onsite installation.

The product has applications throughout the distribution chain. For farms or ponds, the product could be supplied as an onsite installation or as a manufactured product. At farm level, a combination of approaches could be used which includes pre-treatment of the fishes with the product, adding the product to the farm or pond. A similar approach could be used for companies involved in transportation or shipment of Ornamental fishes. The product could be supplied as a manufactured product for owner of Aquariums of different sizes, retail outlets. For huge aquariums, provisions could be done for onsite installation of the technology.

The shelf life of the product depends on the combination of bacteria and media used in the composition and the methodology employed in packaging.

Various objects, features, aspects and advantages of the inventive subject matter will become more apparent from the following detailed description of preferred embodiments, along with accompanying figures.

### DESCRIPTION OF THE DRAWINGS

The accompanying drawings are included to provide further understanding of the present disclosure, and are incorporated in and constitute a part of this specification. The drawings illustrate exemplary embodiments of the present disclosure and together with the description, serve to explain the principles of the present disclosure.

FIG. 1 illustrates an exemplary photograph of Molly treated with different composition(s) for a duration of 9 days. Water wasn't changed for these 9 days, no other additives were used. Fishes were fed twice a day with fish food pellets. Stress factors were aeration stopped for hours, the amount of water used was lesser than prescribed amounts and not changed, the bowl only had an aeration facility and the composition(s) added to the water in the bowl(s). The off-spring of Molly are seen in this image.

FIG. 2, FIG. 3 illustrates an exemplary photograph of the Goldfishes treated with the different composition(s) for a duration of 9 days. Water wasn't changed for these 9 days, no other additives were used. Fishes were fed twice a day with fish food pellets. Stress factors were aeration stopped for hours, the amount of water used was lesser than prescribed amounts and not changed, the bowl(s) only had an aeration facility and the composition(s) added to the water in the bowl(s). Experiment was for 9 days.

FIG. 4 illustrates an exemplary photograph of Koi fishes treated with different composition(s) for a duration of 9 days. Water wasn't changed for these 9 days, no other additives were used. Fishes were fed twice a day with fish food pellets. Stress factors were aeration stopped for hours, the amount of water used was lesser than prescribed amounts and not changed, the bowl(s) only had an aeration facility and the composition(s) added to the water in the bowl(s).

FIG. 5 illustrates an exemplary photograph of Veiltail Angelfishes in transportation bags treated with the composition(s). The bags had 10 Veiltail Angelfishes that survived in the bags for 40 hours and water lesser than prescribed amounts. At the end of 40 hours, no mortality was observed and fishes acclimatized in tanks well and in a short time.

FIG. 6 illustrates an exemplary photograph of Molly with their offspring in the bowl to which the composition(s) were added. Stress factors were aeration stopped for hours, the amount of water used was lesser than prescribed amounts and not changed, the bowl(s) only had an aeration facility and the composition(s) added to the water in the bowl(s).

FIG. 7 illustrates a schematic representation of product as onsite installation and manufactured product.

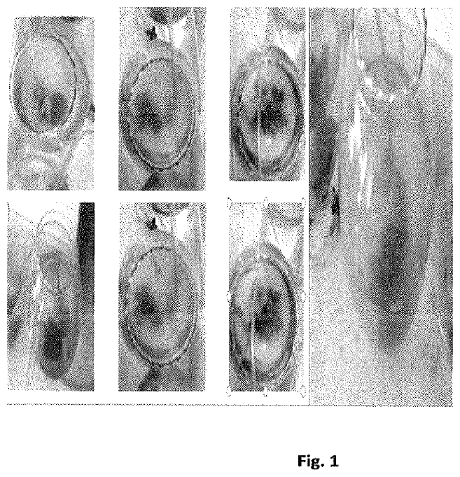

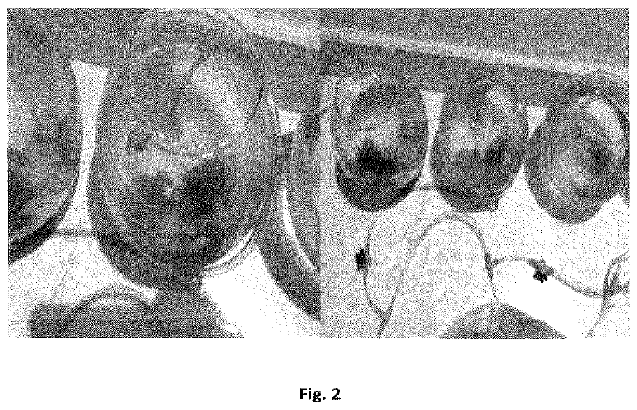

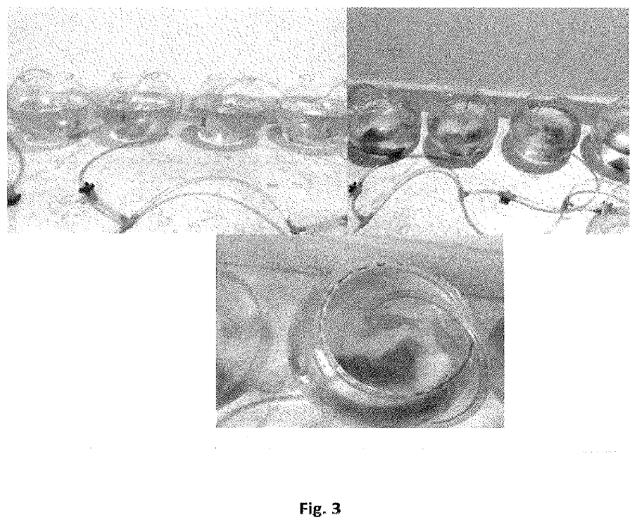

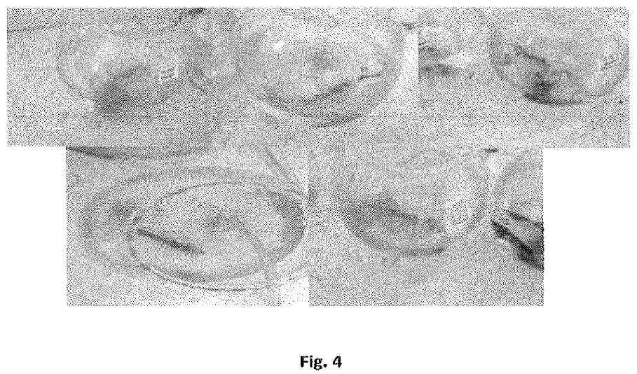

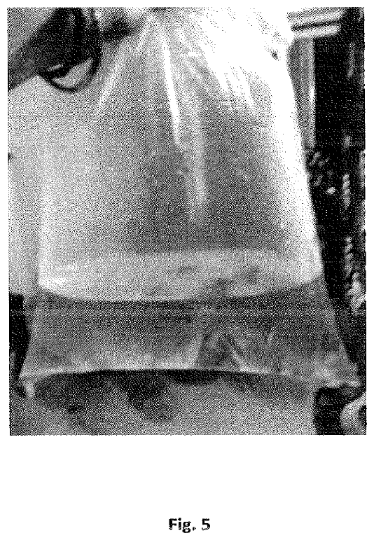

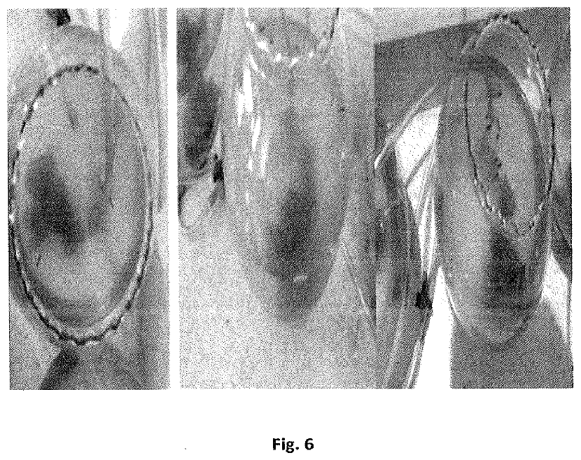

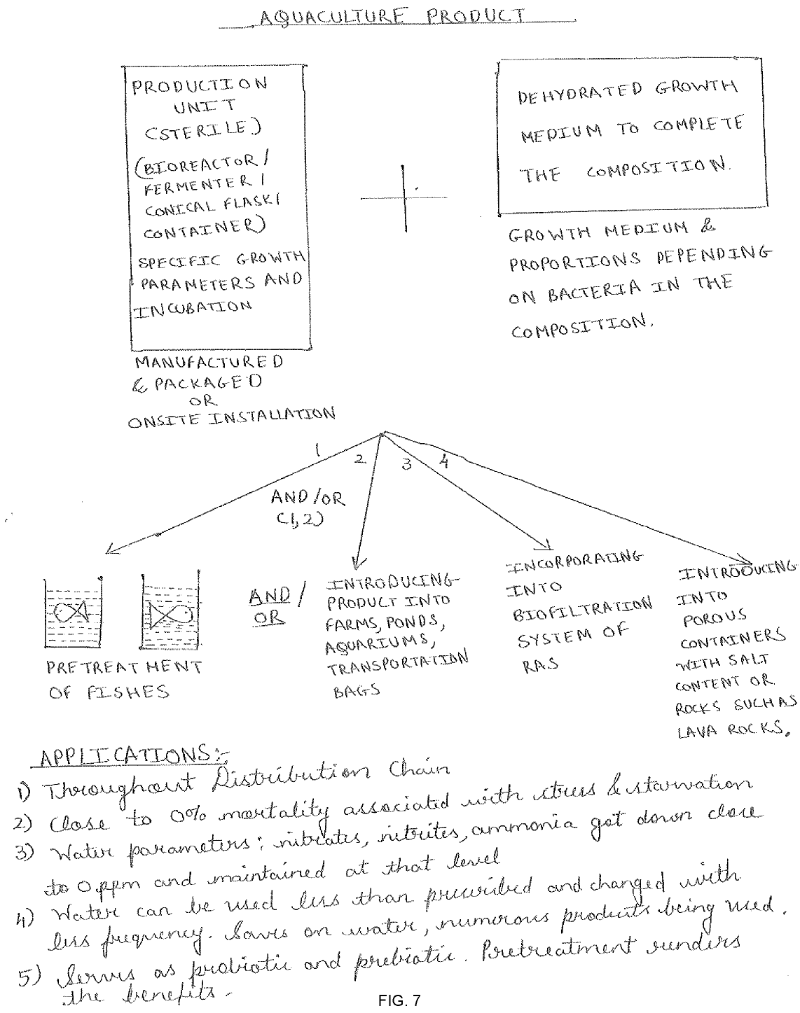

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents and alternatives falling within the spirit and scope of the present disclosure as defined by the appended claims.

Each of the appended claims defines a separate invention, which for infringement purposes is recognized as including equivalents to the various elements or limitations specified in the claims. Depending on the context all references below to the “invention” may in some cases refer to certain specific embodiments only. In other cases it will be recognized that references to the “invention” will refer to subject matter recited in one or more, but not necessarily all of the claims.

As used in the description herein and throughout the claims that follow, the meaning of “a”, “an”, “the” includes plural reference unless the context clearly dictates otherwise. Also, as used in the description herein, the meaning of “in” includes “in” and “on” unless the context clearly dictates otherwise.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Various terms as used herein are shown below To the extent a term used in a claim is not defined below, it should be given the broadcast definition persons in the pertinent art have given that term as reflected in printed publications and issued patents at the time of filing.

There is a wide range of bacteria that could be used in the composition individually or in combination based on the kind of interactions they share and their effect as a probiotic. These include gram positive bacteria and gram negative bacteria.

The bacteria could be New Species, Variants of known species and the known species.

The bacteria belonging to the following genus could be used in the composition. However, this information doesn't restrict the genus of the known species which could be used in the compositIon(s).
Alteromonas, Arthrobacter, Bacillus, Brevibacterium, Hydrogenophaga, Lactobacillus, Leuconostoc, Pediococcus, Phyllobacterium, Pseudoalteromonas, Pseudomonas, Streptomyces, Shewanella.

The species could be as follows but the scope is not restricted to the following and these are representative. The details are as follows:
Alteromonas:
Pseudoalteromonas arabiensis, Pseudoalteromonas luteoviolacea, Pseudoalteromonas rubra, Pseudoalteromonas sp.
Arthrobacter:
Arthrobacter species known to produce therapeutic biomolecules and used based on the fish health condition and fish type.
Bacillus:
Bacillus amyloliquefaciens, Bacillus firmus, Bacillus fiexus, Bacillus lentus, Bacillus pumilus, Bacillus potymyxa, Bacillus megatarium, Bacillus alvei, Bacillus subtilis, Bacillus siamensis, Bacillus sonorensis, Bacillus vallismortis, Bacillus velezensis, Bacillus paralicheniformis, Bacillus vireti
Brevibacterium:
Brevibacterium aureum, Brevibacterium fuscum, Brevibacterium sp.
Hydrogenophaga:
Hydrogenophaga intermedia
Lactobacillus:
Lactobacillus sakei, Lactobacillus rogosae, Lactobacillus plantarum, Lactobacillus pentosus, Lactobacillus paracasei, Lactobacillus panis, Lactobacillus pontis, Lactobacillus reuteri, Lactobacillus fiumenti, Lactobacillus sanfranciscensis, Lactobacillus casei, Lactobacillus coryniformis, Lactobacillus mellis, Lactobacillus brevis, Lactobacillus buchneri, Lactobacillus bulgaricus, Lactobacillus curvatus, Lactobacillus farciminis, Lactobacillus firmentum, Lactobacillus helveticus, Lactobacillis lactis, Lactobacillus reuteri, Lactobacillus johnsonii
Leuconostoc:
Leuconostoc citreum, Leuconostoc pseudomesenteroides, Leuconostoc litchfi
Pediococcus:
Pediococcus acidilactici, Pediococcus damnosus, Pediococcus pentosaceus, Pediococcus cerevisiae, Pediococcus argentinicus, Pediococcus cellicola, Pediococcus claussenii, Pediococcus ethanolidurans, Pediococcus inopinatus Pediococcus parvenus, Pediococcus pentosaceus, Pediococcus siamensis, Pediococcus stilesii
Phyllobacterium:
Phyllobacterium brassicacearum, Phyllobacterium ifriqiyense, Phyllobacterium sophorae, Phyllobacterium catacumbae, Phyllobacterium endophyticum, Phyllobacterium ifriqiyense, Phyllobacterium leguminum, Phyllobacterium loti Phyllobacterium myrsinacearum, Phyllobacterium phragmitis, Phyllobacterium rubiacearum, Phyllobacterium salinisoli, Phyllobacterium sophorae, Phyllobacterium trifolii, Phyllobacterium zundukense
Pseudoalteromonas:
Pseudoalteromonas arabiensis, Pseudoalteromonas luteoviolacea, Pseudoalteromonas rubra
Pseudomonas:
Pseudomonas alkylphenolica, Pseudomonas alcaliphila, Pseudomonas bauzanensis, Pseudomonas clemancea,-Pseudomonas chlororaphis, Pseudomonas carboxydohydrogena, Pseudomonas glycinae, Pseudomonas koreensis Pseudomonas kilonensis, Pseudomonas indica, Pseudomonas lini, Pseudomonas rhizosphaerae, Pseudomonas pachastrellae, Pseudomonas grimontii, Pseudomonas graminis
Shewanella:
Shewanella sediminis
Streptomyces:
Streptomyces violaceoruber, Streptomyces ansochromogenes, Streptomyces aureofaciens, Streptomyces noursei Streptomyces rimosus, Streptomyces sp. S073

New species of bacteria belonging to the family Pseudomonadaceae that render all the characteristics of the composition(s) mentioned here were isolated. These bacteria based on biochemical characteristics, Antibiotic Sensitivity Testing (AST), Whole Genome Studies using NGS, could be classified under the family Pseudomonadaceae. The whole genome sequences of these isolates were found to match to the known species of Pseudomonas within the range of 75%-85%.

Each of the bacteria in the composition(s) used either individually or in combination(s) in the composition(s) were found to be highly beneficial for fishes.

One example of growth medium is Nutrient Broth and its dehydrated form added to the incubated bacterial culture to prepare the composition(s). The bacteria used in the composition(s) were incubated in growth media for a minimal time of 48 hours before being used in the composition(s). The bacterial suspensions over cell pellets was preferred in preparing the composition(s). Growth media was added in a dehydrated form. However, there could be alterations in the proportions of the growth media used. The growth media could be altered considering the incubation time of the bacteria being used in the composition(s). Also, the bacterial cell mass instead of suspension could be used in preparing the composition(s).

The growth parameters have a vital role in preparing the composition(s) that could render the characteristics mentioned in this specification.

Growth medium: The growth media could be simple, complex, selective, customized media. The optimal medium for a bacteria to be used in the composition could be decided based on the growth pattern studies. In the methodology used, the same medium in dehydrated form could be added to the suspension culture or a different medium dehydrated powder.

The incubation time depends on the inoculum, preference for the media and the growth parameters chosen.

The process or method for preparing the composition(s) involves taking aliquots of the bacterial suspensions incubated for a particular duration of time (minimum of 48 hours) and adding the growth media, mixing for about 1-5 minutes and then introducing into tanks, farms or transportation bags for fishes. A volume of 1 ml of the composition(s) was used for 1 litre of water. So, for example, if a fish was in a tank with 1 litre of water, then 1 ml of the composition(s) was added. If the tank had 5 litres of water, then 5 ml of the composition(s) was used. This is applicable for all the composition(s) and the simplest process or method, there could be variations to this process or method. Variations could be in the composition(s) being used and the amount of the composition(s) being used. Above mentioned process is only an example and there could be numerous variations to the same.

The methodology of usage of the composition(s) is environment specific, in other words if the composition(s) is being used at aquariums, during transportation, at farms or ponds, retail outlets or end users. In case of farms or ponds, the composition(s) could be introduced into the farms or ponds, used to pretreat the fishes and preferably a combination of both. For water quality, the addition of the composition(s) into the farms or ponds and the composition(s) incorporated into the bio filtration system of RAS helps get the water parameters such as nitrates, nitrites and ammonia close to 0 ppm and maintain at that level. In the case of aquariums, during transportation, retail outlets and end users either of the methodologies could be used or both the methodologies could be implemented.

There could be variations in this process or method in terms of the form in which the bacteria is being used in the composition(s)—either as cell mass or suspensions, the incubation times chosen for the bacteria used in the composition(s), the proportions of growth media added to the bacterial suspension, the kind of growth media being used and it could be a custom growth media composition(s) as well, the combination(s) of bacteria being used in the composition(s). The bacteria could be used in the composition either individually or in combination(s), in the composition(s).

To elaborate further, variations could be there considering the various growth parameters for the bacteria, their incubation times and the type of fishes for which the composition(s) are being prepared, the kind of growth media chosen, the nutritive media chosen, the proportions of the bacteria used in the composition(s) in permutations or combinations or if composition(s) are prepared using the individual bacteria, whether the bacteria are used as suspension cultures or as cell pellets, the extent of role of the composition(s) in cleansing water and serving as probiotic and prebiotic taking into account the water quality to be addressed, the density of fishes, fish type, their food feed patterns.

The above mentioned composition(s) served as probiotics for fishes. With inclusion of nutritive media(s), the composition(s) could serve as prebiotics as well. The growth medium could be customized to have the components that serve as prebiotics. Also, with modulation of proportions of growth media, we could monitor the extent of growth of bacteria in the water, which enables improve water quality by reducing nitrates, nitrites and ammonia close to 0 ppm and maintaining at that level.

The various benefits observed using the various composition(s) were close to 0% mortalities throughout the distribution chain of Aquaculture industry, nitrates, nitrites and ammonia were got down close to 0 ppm and maintained at that level, disease control, improvement in digestion, improvement in immune system, improvement in color, more compatible with other fishes, serves as source of nutrients, growth promotor, improves water quality, water needs to be changed with lesser frequency, water used can be lesser than the currently prescribed amounts, promotes reproduction.

The invention comprises of systems. These include Systems to achieve close to 0 ppm of nitrates, nitrites and ammonia at aquariums and Fish Only Tanks, Systems to incorporate the composition(s) into biofiltration system of Recirculatory Aquaculture Systems (RAS), Systems to enable the production unit of composition(s) to be an integral setup of farms.

The system(s) to attain close to 0 ppm of nitrates, nitrites and ammonia at aquariums could be rocks such as lava rocks into which the composition(s) could be introduced. Alternatively, it could comprise of containers such as spherical porous containers into which pieces of rocks, stones (these are examples) could be placed. The composition(s) are introduced into this container and then this container could be placed in Fish Only Tanks or aquariums. While rocks can be placed in an aquarium and improves aesthetic value, for Fish Only Tanks, the system of spherical perforated container mentioned is preferred. In the case of farms, the composition(s) could be incorporated into the biofiltration system of Recirculatory Aquaculture System (RAS). System(s) could be custom designed to enable get nitrates, nitrites and ammonia close to 0 ppm throughout the distribution chain of the Aquaculture industry.

Systems to enable the production unit of composition(s) to be an integral setup of farms, aquariums: This system for example could be a bioreactor setup with outlets connected to the farms or aquariums. Custom designed systems could be used for the production unit(s) to be placed at farms or huge aquariums or at companies involved in shipments of huge quantities of the livestocks.

The mortalities were got down considerably to 0% at aquariums and during transportation (DOA—Death On Arrival, DAA—Death After Arrival, DA7 —Death After 7 days Of Arrival) on using these composition(s). This is applicable throughout the distribution chain of the Aquaculture industry. Water parameters such as nitrates, nitrites and ammonia were got down close to 0 ppm and maintained at that level, using the above mentioned composition(s). Water needed to be replaced with lesser frequency and could remain unchanged for around 2 weeks. The packaging of fishes could be done using lesser than prescribed amounts of water. The invention serves as probiotic, prebiotic and improves the water quality.

The invention addresses the stress and starvation associated with fishes.

Different composition(s) were used and in different proportions for this study and some proportions of composition(s) suited best for a particular type of fishes. However, all the bacterial composition(s) had the benefits described in the invention for all the fishes; fine tuning the proportions of the composition(s) customized these composition(s) for each of the fish type.

The methodology of usage of the composition(s), method(s), system(s) could be altered based on the environment where it is being used. The following are only instances and do not restrict the scope of methodologies that could be used in the different environments. Some of the instances are as follows:

Farm/Pond:
The composition(s) could be used to pre-treat the fishes, fry, fingerlings before they are introduced into the farm or pond. The duration, composition, method, system required for this pretreatment could vary and is also dependent on the fish type and stage of growth. The water treatment could be attained by incorporation of this composition(s) into Recirculatory Aquaculture System (RAS) or by treating the water in the bio pond or treatment pond. The water treatment could be attained even while the aquatic life forms are there in the pond or farms.
Alternative approach is to add the composition(s) directly into the farm/pond.
Which method is feasible depends on the farm size, density of fishes, types of fishes, kind of food fed, level of water purity.
System(s) are required to improve the water quality.
The composition(s), method(s), system(s) need to be altered based on the site being treated with this product.
The product could be available as an onsite installation or provided as a manufactured product.

The composition(s) could be introduced to the pond or farm at the intervals of a fortnight which could include independently treating the fishes and water, pretreatment of fishes or introducing to the pond/farm that would involve treating the fishes and water together. The interval and method of introduction of the composition(s) to the farm/pond depends on the size of the farm/pond, type of aquatic life forms, density of live stock, water quality and health parameters.
The composition(s), method(s), system(s) could be used to improve water quality of freshwater, marine water.
While in clean water the composition(s), method(s), system(s) enables maintain the water quality, serves as probiotic and prebiotic and when used in water with poor quality it renders the benefits of improving the water quality apart from the benefits of probiotic and prebiotic. The product essentially addresses stress and starvation and hence renders the numerous benefits.
The composition(s), method(s), system(s) work at optimal in freshwater and marine water environments and there is no difference in efficacy observed when these two environmental conditions are compared.
Aquariums:
The composition(s), method(s), system(s) could be used in the aquariums with very high nitrates, nitrites, ammonia and turbid water. The product gets the nitrates nitrites, ammonia close to 0 ppm and maintains at that level. Further, the water becomes clearer and fit for live stock. In other words, the product helps bioremediation of water making it re-usable for aquaculture. This was attained in a short duration-in 96 hours. Apart from improving water quality, the benefits of probiotic were observed, which includes: Promotor of reproduction, Disease control, Improvement of digestion, Improvement in immune system, Source of nutrients and growth promotor, Improvement in color, more compatible with other fishes. The product also serves as a prebiotic and this depends on the growth or nutritive medium being used in the composition. In this study, the water treatment to make it fit for livestock took about 96 hours but the live stock in this aquarium were found to have improved health and no mortality even before the water was made totally fit for aquaculture.
The composition(s), method(s), system(s) could be used in clean freshwater, clean marine water, freshwater or marine water not in optimal conditions.
While in clean water the composition(s), method(s), system(s) enables maintain the water quality, serves as probiotic and prebiotic and when used in water with poor quality it renders the benefits of improving the water quality making the same fit for aquaculture and also renders the benefits of probiotics and probiotics. The product essentially addresses stress and starvation and hence renders the numerous benefits.
Composition(s) could be introduced to the aquarium at the intervals of a fortnight which could include independently treating the fishes and water, pretreatment of fishes or introducing to the aquarium that would involve treating the fishes and water together. The interval and method of introduction of the composition(s) to the farm/pond depends on the size of the farm/pond, type of aquatic life forms, density of live stock, water quality and health parameters.
The composition(s), method(s), system(s) work at optimal in freshwater and marine water environments and there is no difference in efficacy observed when these two environmental conditions are compared.
For huge aquariums, onsite installation is possible. Alternatively, for all size and kind of aquariums, the product could be supplied as a manufactured product.

Transportation of Live Stock:
The fishes could be pretreated with the composition(s) and then introduced to a transportation bag without the composition(s) introduced to this bag OR,
The fishes could be pretreated with the composition(s) and then introduced to a transportation bag with the composition(s) introduced to this bag OR,
The fishes could be introduced to a transportation bag with the composition(s) introduced to this bag and no pretreatment done.

Which of the above mentioned method is chosen, depends upon the kind of fish, fish density being transported, water used for transportation, duration of travel and fish health condition.

Retail outlet and end users aquariums: The usage is in lines of the specification provided in point [0072] for Aquariums. However, for end users providing manufactured product is preferred.

We could select numerous bacteria to be included as part of the composition(s) and not necessarily the bacteria with the Accession Numbers mentioned in this specification.

Onsite installation procedure(s) for the production and usage of composition(s), with the required method(s) and system(s) are as follows:

Onsite installation at Farm/Pond:

This would require a setup of production unit onsite, near the farm or pond. The operations and maintenance needs to be taken care by a qualified microbiologist. The parameters set for the production are vital to cater to the methodologies of preparation of the composition(s). The size of the production unit depends on the size of the farm/pond, method of treatment process to be implemented, fish type, fish density, fish health, fish food, water quality, pretreatment procedures.

The system(s) used onsite vary in size and numerous system(s) could cater to this process. What size, material that is used, number of such system(s) which are used is dependent upon the size of the farm/pond, size of fish, type of fish and water quality.

The onsite installation could be for the pretreatment of the fishes, treatment of water to improve water quality and introducing into the farms, ponds. Which approach is taken depends on fish type, fish density, fish health, water quality, fish food, size of the pond/farming treated.

The onsite installation could be used for treating the fishes and the water quality by using the composition(s), method(s), system(s) in the pond/farm.

The method of onsite treatment purely depends on the fish type, fish density, fish food, water quality, size of the pond/farm. Alternate to onsite installation is providing the manufactured product. The packaging could be done using recyclable products, containers and the shelf life depends on the components of the composition; the best to use time racing from one month to six months.

Onsite installation for Aquariums:
This is in the case of aquariums of huge size, examples include S.E.A. Aquarium,—Singapore, Georgia Aquarium—USA, Dubai Aquarium and underwater Zoo—UAE, AquaDom Sea Life, Germany.
Here, the product could be supplied as a manufactured product as refillable cans (or similar packaging systems) or have onsite installation facility. The specification of onsite installation is similar to that mentioned for ponds/farms.

For retail outlets and end users, the product could be supplied in disposable cans or similar such packaging.

Onsite installation for Companies into Transportation of Live stock:
The onsite installation here is defined as the pre-treatment facility for fishes or Supplying manufactured product to be added to transportation bags or Refillable cans or similar packaging onsite for the product to be used or Onsite installation similar to that done for farms/ponds/aquariums.

In case the bioreactor/fermenter is used onsite, the parameters set for the production have a crucial role to play in the manufacturing of the product which has the benefits mentioned in this specification.

The product could be provided as a manufactured product with clear instructions about the way the component(s) of the composition(s) need to be mixed and its usage.

The composition(s), system(s) could be made available as an onsite installation or as a manufactured product.

The packaging could be done using recyclable material, containers of different capacities depending on the volume of the product required to treat an environment. The shelf life of the product could be found by studying the growth curve patterns of the bacteria, ingredients used in the composition(s).

### EXAMPLES

### Example 1

Using the composition(s), method(s), system(s), the mortalities of fishes were found close to 0%: Death On Arrival (DOA), Death After Arrival (DAA), Death After 7 days Of Arrival (DA7) were maintained close to 0%. Also, the mortalities were maintained close to 0% in the bowl where the fishes were subjected to stressful conditions. Close to 0% mortality could be maintained throughout the distribution chain of aquaculture industry:

Some of the representative fishes for the study were Veiltail Angel fishes, Koi, Goldfish, Molly, Cat fishes. They were packaged with water far lesser in amount than the one prescribed in the protocols. The amount of water used for containing 4-5 fishes of Molly, 2 of Goldfishes, 4 of Veiltail Angel fishes, 2 of Koi were 600 ml-1000 ml. Catfish was placed in a bigger container with about 3 litres of water. The fishes could survive the stressful conditions, when the composition(s) were used.

The stressful conditions mainly were: not changing water for 9 days, using lesser than prescribed amounts of water, stopping aeration for hours, fish feed once or maximum twice a day, only few pellets used as feed. The modes of transportations were road ways and shipment (airways). No mortalities were observed.

These fishes were studied at aquariums, considering the same set of stress factors and the various composition(s).

Different proportions of the composition(s) were used and some proportions were best suited for particular type of fishes. Pre-treatment of fishes with the composition(s) before transportation too helped in maintaining DOA, DAA, DA7 close to 0%.

There were Veiltail Angel fishes, Angel fishes packaged in a transportation bag one less in size than the one prescribed in the protocols for packaging. Water used was much less than the prescribed amounts. The fishes were transported by air, shipment time nearly 40 hours, no mortalities were observed.

There were no additives used in these experiments other than the formulation(s). The composition(s) used in this study were New Species belonging to the family Pseudomonadaceae. There could be varied nutritive additives which could be used to further serve as prebiotic. The fishes described here are only representative and the invention is applicable for all fishes.

### Example 2

Composition(s), method(s), system(s) eliminates stress and starvation associated with the fishes: The fishes were placed in a bowl with water close to 600 ml-1 litre, two fishes per bowl, water not changed for 9 days, aerations stopped for hours and fish feed in minor amounts, feed twice a day and sometimes once a day. The composition(s) were added to these bowls. There were controls for the experiment. Koi fishes are representative examples and the invention is applicable for all fishes.

One indicator of stress levels could be the Blood lactate levels. Koi fishes were treated with different composition(s) and there were controls. Only the Koi fishes treated with these composition(s) survived and their blood lactate levels (one of the indicators of stress) was estimated to be at 0.2 mmol/L. Certain composition(s) in certain proportions were best suited for this stressful condition. The bacteria used in these studies were New Species belonging to the family Pseudomonadaceae. The control fishes couldn't survive these stressful conditions. There are numerous other parameters to quantitatively express stressful conditions such as blood parameters.

This confirms the efficacy of the invention for the numerous benefits described in this application. The Koi fish mentioned here is only a representative example. Also, the proportion(s) of the components of the composition(s) used in this example is representative and a range of proportion(s) of different permutations and combinations could be used for the composition(s). The invention isn't restricted to only these fishes and are applicable for all fishes.

### Example 3

The composition(s), method(s), system(s) reduce nitrates, nitrites and ammonia build-up and maintain at low ppm, close to 0 ppm. This enables usage of lesser amount of water during transportation and water needs to be replaced with lesser frequency.

Th nitrates, nitrites and ammonia levels in water are got down to low levels, close to 0 ppm and maintained at that level. This involves usage of the composition(s), system(s) and method(s). The system could comprise of materials like lava rocks which is porous. The composition(s) could be added to this rock and then placed into the aquarium. In the case of Fish Only Tanks, the system could be varied. For example, a spherical container with perforations, inside which densely packed rocks are placed (rock is an example) and the composition(s) is added to these containers. For farms, the composition(s) could be integrated into the Recirculatory Aquaculture System (RAS) to improve the water quality, by reducing nitrates, nitrites and ammonia close to 0 ppm and maintaining at that level.

The representative fishes for the study includes Koi Fishes, Angel fishes, Goldfishes, Molly, Cat fishes. These fishes were observed for their ability to survive in the environment, in presence of high nitrates, nitrites and ammonia in the presence of various composition(s) and without addition of the composition(s). Fishes could survive the stressful conditions only in the presence of composition(s). The method(s) and system(s) were chosen appropriately depending on the environment being treated and the efficacies can be observed throughout the distribution chain of the Aquaculture industry. The fishes mentioned in this example are only representative and the composition(s), method(s), system(s) benefits are applicable throughout the distribution chain of the Aquaculture industry.

### Example 4

Acclimatize in new environments in a short duration: The fishes following transportation, if introduced into tanks with composition(s); the time taken for adjustment is negligible. In the case of fishes pre-treated with the composition(s), the fishes following transportation could be introduced in tanks even without the composition(s) added to the tanks. The representative fishes for the study includes Koi Fishes, Angel fishes, Goldfishes, Molly, Cat fishes. The invention isn't restricted to only these fishes and are applicable for all fishes.

### Example 5

### An illustration of how the proportions of the components of the composition(s) could be altered:

The basic example of preparing composition(s) is using 48 hour old incubated bacterial cultures. The culture suspension is only one of the component of the composition(s). To this, growth media which is preferable for the bacterial growth is added. An instance is using 1 ml of suspension culture to which 0.013 gms of dehydrated Nutrient broth powder is added. This composition prepared is to be used for 1 litre of water. In case of 5 litres of water, 5 ml of composition could be used. This is just the very basic example and variations could be there considering the various growth parameters for the bacteria, their incubation times and the type of fishes for which the composition(s) are being prepared, the kind of growth media chosen, the nutritive component chosen, the proportions of the bacteria used in the composition(s) in permutations or combinations or if composition(s) are prepared using the individual bacteria, whether the bacteria are used as suspension culture or as cell pellet, the extent of role of the composition(s) in improving water quality, serving as probiotic and prebiotic, the density of fishes, fish type, their food feed patterns. The probiotic bacteria other than the ones whose Accession Numbers have been mentioned in this specification too could be used to prepare the composition(s) to attain the various benefits characteristic of the composition(s), method(s), system(s).

### Example 6

The composition(s) serve as Probiotic for fishes: The composition(s) used with the appropriate method(s), system(s) led to improved color in fishes, better appetite, improved health as was observed with the various blood parameters, fishes found to be more compatible with other fishes and less aggressive, reduced stress and starvation, which in turn improved the longevity of the fishes. Different proportions of the composition(s) were used and some proportions were best suited for particular type of fishes.
Pre-treatment of fishes with the composition(s) helped in achieving the benefits of the composition(s) as a probiotic.
The representative fishes for the study includes Koi Fishes, Angel fishes, Goldfishes, Molly, Cat fishes. The invention isn't restricted to only these fishes and are applicable for all fishes.

### Example 7

The composition(s) serve as Prebiotic for fishes and enables improve water quality: The composition(s) could serve as a prebiotic and improves the water quality. The nutritive additive plays the role of prebiotic and the growth media for proliferation of bacteria cells in the water, that enable improve water quality. The proportions of the bacteria, their incubation bation times, the growth parameters of the bacteria, the permutations and combinations of the bacteria, the proportions of growth media, the form in which growth media is used, which growth media was used; all these parameters could be modulated to address the kind of fishes and the environment in which the composition(s), method(s), system(s) are being used. The representative fishes for the study includes Koi Fishes, Angel fishes, Goldfishes, Molly, Cat fishes. The invention isn't restricted to only these fishes and are applicable for all fishes.

### Example 8

Fish breeding observed with the composition(s), method(s), system(s): The representative example mentioned here is Molly. An example of the composition used here is formulation comprising of New Species of bacteria and Nutrient broth. After completion of 28 hours, lava rocks were added to the bowls. The 4 Mollies treated with the composition gave birth to 8 offspring. The composition used comprised of New Species of bacteria belonging to the family Pseudomonadaceae.

### Example 9

Production of the composition(s): With the easy to scale up procedures for the production of the composition(s), these composition(s) could be made an integral setup of aquaculture farms and throughout the distribution chain. The product could be manufactured to cater to the distribution chain of the aquaculture industry. The fishes mentioned in the examples are only representative and the scope of the invention isn't restricted only to these fishes.

### Example 10

### Pond, farm or any water body/environment suitable for aquaculture:

The fishes and aquatic life forms could be pretreated with the composition(s) in a prescribed manner and then introduced into the pond or water bodies/environment. The product could be directly introduced into the water body/environment. A combination of approaches could be used based on the water environment, size of the water environment, aquatic life forms, water quality. The system(s) to be used are environment specific.

### ADVANTAGES OF THE INVENTION

The present disclosure relates to composition(s), method(s), system(s) to address stress, starvation, improved health, improved water quality, productivity and life expectancy for fishes. The product addresses the concerns associated with Aquaculture industry, giving better living conditions for the fishes and eliminates the kind of stress, starvations they would face throughout the distribution chain of the Aquaculture industry. The product serves as probiotic, prebiotic, improves water quality. Also, with this product the water used at aquariums needs to be replaced with lesser frequency and amount of water used for transportation could be lesser than the currently prescribed amounts in the protocols. These features mentioned are the major characteristics of the composition(s) and each of the composition(s) address the various health parameters associated with each type of fishes. The product has applications throughout the distribution chain, starting from farms, warehouses, retailers, end users. The production unit of the composition(s) could be made an integral setup of Aquaculture farms,

The product eliminates stress and starvation associated with fishes,

The product enables close to 0% mortalities during transportation; Death On Arrival (DOA), Death After Arrival (DAA), Death After 7 days Of Arrival (DA7) are maintained close to 0%. Also, the mortalities throughout the distribution chain are maintained close to 0%,

The product serves as probiotic to fishes,

The product serves as prebiotic,

The product reduces nitrates, nitrites and ammonia build-up and maintain at low ppm, close to 0 ppm, thus improving water quality,

The fishes could be pre-treated with the product for the various benefits rendered to the fishes,

The product enables water to be replaced with lesser frequency at farms, aquariums and packaging for transportation can happen with water lesser than the amount prescribed in existent protocols,

The product could be incorporated into Recirculating Aquaculture System (RAS),

The product have varied effects on the different types of fishes. The composition(s) could be fine tuned in terms of the proportions of the components of the composition(s),

The product enables fishes to acclimatize at aquariums in a very short time,

The production unit for the product could be made an integral setup of farms.

1. Onsite production or supply as Manufactured product of the composition(s) to the Aquaculture industry distribution chain comprising of:
Bacteria that could be New Species,
Bacteria that could be a variant of the known species,
Bacteria that are known species belonging to a range of genera,
Bacteria that are known species and belong to the following representative genus:
Alteromonas, Arthrobacter, Bacillus, Brevibacterium, Hydrogenophaga, Lactobacillus, Leuconostoc, Pediococcus, Phyllobacterium, Pseudoalteromonas, Pseudomonas, Streptomyces, Shewanella.
Bacteria belonging to the representative species:
Pseudoalteromonas arabiensis, Pseudoalteromonas luteoviolacea, Pseudoalteromonas rubra, Pseudoalteromonas sp., Bacillus amyloliquefaciens, Bacillus firmus, Bacillus flexus, Bacillus lentus, Bacillus pumilus, Bacillus polymyxa, Bacillus megatarium, Bacillus alvei, Bacillus subtilis, Bacillus siamensis, Bacillus sonorensis, Bacillus vallismortis, Bacillus velezensis, Bacillus paralicheniformis, Bacillus vireo, Brevibacterium aureum, Brevibacterium fuscum, Brevibacterium sp., Hydrogenophaga intermedia, Lactobacillus sakei, Lactobacillus rogosae, Lactobacillus plantarum, Lactobacillus pentosus, Lactobacillus paracasei, Lactobacillus panis, Lactobacillus pontis, Lactobacillus reuteri, Lactobacillus frumenti, Lactobacillus sanfranciscensis, Lactobacillus casei, Lactobacillus coryniformis, Lactobacillus mellis, Lactobacillus brevis, Lactobacillus buchneri, Lactobacillus bulgaricus, Lactobacillus curvatus, Lactobacillus farciminis, Lactobacillus fermentum, Lactobacillus helveticus, Lactobacillis lactis; Lactobacillus reuteri, Lactobacillus johnsonii, Leuconostoc citreum, Leuconostoc pseudomesenteroides, Leuconostoc litchii, Pediococcus acidilactici, Pediococcus damnosus, Pediococcus pentosaceus, Pediococcus cerevisiae, Pediococcus argentinicus, Pediococcus cellicola, Pediococcus claussenii, Pediococcus ethanolidurans, Pediococcus inopinatus, Pediococcus parvenus, Pediococcus pentosaceus, Pediococcus siamensis, Pediococcus stilesii,
Phyllobacterium brassicacearum, Phyllobacterium ifriqiyense, Phyllobacterium sophorae, Phyllobacterium catacumbae, Phyllobacterium endophyticum, Phyllobacterium ifriqiyense,
Phyllobacterium leguminum, Phyllobacterium loci, Phyllobacterium myrsinacearum, Phyllobacterium phragmitis, Phyllobacterium rubiacearum, Phyllobacterium salinisoli, Phyllobacterium sophorae, Phyllobacterium trifolii, Phyllobacterium zundukense, Pseudoalteromonas arabiensis, Pseudoalteromonas luteoviolacea, Pseudoalteromonas rubra, Pseudomonas alkylphenolica, Pseudomonas alcaliphila, Pseudomonas bauzanensis, Pseudomonas clemanceaPseudomonas chlororaphis, Pseudomonas carboxydohydrogena, Pseudomonas glycinae, Pseudomonas koreensis, Pseudomonas kilonensis, Pseudomonas indica, Pseudomonas lini, Pseudomonas rhizosphaerae, Pseudomonas pachastrellae, Pseudomonas grimontii, Pseudomonas graminis, Shewanella sediminis,
Streptomyces violaceorubm; Streptomyces ansochromogenes, Streptomyces aureofaciens, Streptomyces noursei, Streptomyces rimosus, Streptomyces sp. S073, Arthrobacter species known to produce therapeutic biomolecules and used based on the fish health condition and fish type.
Growth medium could be simple, complex, selective or customized,
The dehydrated Growth medium added after incubating the bacteria could be the same medium used for the cultivation of bacteria or a different Growth medium,
The dehydrated Growth medium added could be proportional to the amount specified for a given volume, as given in the instructions by the manufacturer of the media or could be customized,
There could be variations to this basic rule in the method of preparation depending on where it is being used, bacterial proportions and density, fish taxonomy, water quality, fish density,
Variations in the composition(s) and their proportions in permutations and combinations and along with the system(s) and method(s) renders the various benefits characteristic of the composition(s).

2. The composition as recited in claim 1, wherein the incubation time depends on the bacteria, growth medium used, preference for the growth medium studied using growth patterns and inoculum density. Generally, a minimum incubation of 48 hours is preferred and the growth conditions dependent on the bacteria used in the composition(s).

3. The composition as recited in claim 1, wherein the shelf life of the composition(s) depends on the bacteria, growth medium used in preparation of the composition(s) and the best to use time could range from a month to six months from the date of manufacturing.

4. The composition as recited in claim 1, wherein the bacteria could be a probiotic bacteria.

5. The distribution chain as recited in claim 1 comprises of the farms, ponds, aquariums, transportation bags, retail outlets, end users.

6. The composition of claim 1, wherein the composition(s) could be fine tuned for a specific fish type. The composition of claim 1, wherein the composition(s) could be highly specific for a fish type or fish taxonomy.

7. The composition(s) as recited in claim 1, the method(s) and system(s) for aquaculture industry distribution chain characterized by:
Addressing the stress and starvation of fishes throughout the distribution chain,
Enabling close to 0% mortalities of fishes throughout the distribution chains; at farms, at aquariums, during transportations (Death On Arrival—DOA, Death After Arrival—DAA, Death After 7 Days Of Arrival—DA7), retail outlets and end users,
Pre-treatment of fishes to achieve close to 0% mortalities of fishes throughout the distribution chains; at farms, at aquariums, during transportations (Death On Arrival DOA, Death After Arrival—DAA, Death After 7 Days Of Arrival—DA7), retail outlets and end users,
Improving the health parameters of the fishes,
Serving as a probiotic,
Serving as a prebiotic,
Serving to improve water quality by reducing high levels of nitrates, nitrites and ammonia close to 0 ppm and maintaining at that level,
Promotor of reproduction,
Disease control,
Improvement of digestion,
Improvement in immune system,
Source of nutrients and growth promotor,
Improvement in color,
More compatible with other fishes,
Usage of water lesser than the currently prescribed amount and replacing water with lesser frequency,
Enabling immediate acclimatization to the new environment the fishes are introduced,

8. The composition as recited in claim 1, wherein the composition(s) could comprise of one type of species of bacteria or a combination of them, which could be New species, variants, known species.

9. The composition as recited in claim 1, wherein the composition(s) comprise of growth media which are chemically defined or customized.

10. The composition as recited in claim 1, wherein the composition(s) comprise of known nutritive components that could serve as a prebiotic or customized.

11. The composition as recited in claim 1, wherein the growth media could also serve as the prebiotic nutritive components.

12. The composition as recited in claim 1 wherein the growth media used is proportional to the amount to be used for a given volume, as given in the instructions by the manufacturer of the media or customized.

13. The method of claim 7, wherein the method is environment specific and also depends on the fish type, the quality of water, density of fishes, fish feed patterns.

14. The method of claim 14 wherein the method for farms, ponds, aquariums, transportation bags could be pretreatment of fishes and introducing the composition(s) directly into the ponds, farms, aquariums, tanks, transportation bags.

15. A method of claim 7, wherein the method comprises of taking aliquots of the bacterial suspension incubated for a minimum of (24-48) hours, adding the dehydrated growth medium and mixing for about 1-5 minutes and then introducing into tanks, aquariums, farms, ponds, transportation bags; volume of (1-5) ml of the composition(s) use for one liter of water

16. A method of claim 7 wherein the composition(s) could be injected into the fishes.

17. The system for method recited in claim 15 wherein the system to get down water parameters-nitrates, nitrites and ammonia close to 0 ppm and maintain at that level could be a porous containers of different shape and size with material having high salt content.

18. The system of claim 16, wherein the material used in the porous containers could be lava rocks.

19. The system for method recited in claim 15 wherein the system to get down water parameters—nitrates, nitrites and ammonia close to 0 ppm and maintain at that level is to introduce the composition(s) into the Recirculatory Aquaculture Systems (RAS).

20. The onsite installation of the composition(s) as recited in claim 1 requires customized solutions catering to the specific needs of the farms, ponds, aquariums, or companies into transportation of live stock—Ornamental fishes.

21. (canceled)
